(19) United States
(12) Patent Application Publication
(10) Pub. No.: US 2009/0176849 A1
(43) Pub. Date: Jul. 9, 2009

(54) Process for the preparation of 2-alkyl-1-((2'-substituted-biphenyl-4-yl) Methyl)-imidazole, dihydroimidazole or benzimidazloe derivatives

(75) Inventor: Lovro Selic, Celje (SI)

Correspondence Address: ARENT FOX LLP, 1050 CONNECTICUT AVENUE, N.W., SUITE 400, WASHINGTON, DC, 20036, US

(73) Assignee: LEK PHARMACEUTICALS, D.D., Ljubljana (SI)

(21) Appl. No.: 11/920,870

(22) PCT Filed: May 22, 2006
(86) PCT No.: PCT/EP2006/004843

§ 371 Date: Feb. 9, 2009

(30) Foreign Application Priority Data
May 24, 2005 (SI) P200500155

(51) Int. Cl. A61K 31/41; C07D 403/06; A61K 31/4184; C07D 403/04

(52) U.S. Cl. 514/382; 548/250; 514/394; 548/305.4

(57) ABSTRACT

The invention relates to a new process for the preparation of sartans 2-butyl-3-[[2′-[1-(triphenylmethyl)-1H-tetrazol-5-yl][1,1′-biphenyl]-4-yl]methyl]-1,3-diazaspiro[4.4]non-1-en-4-one is disclosed, which proceeds via novel intermediate, 4-[(2-butyl-4-oxo-1,3-diazaspiro[4.4]non-1-en-3-yl)methyl]phenylboronic acid (Formula (II)) or its analogs. Compound (II) reacts with 5-(2-bromophenyl)-1-(triphenylmethyl)-1H-tetrazole (III) in the presence of catalyst, using conditions of Suzuki reaction, to give trityl irbesartan (I), whereas analogs to compound (II) may give candesartan, valsartan, telmisartan, losartan and olmesartan.

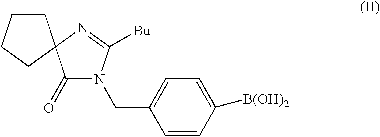

### FIELD OF THE INVENTION

The invention belongs to the field of organic chemistry and relates to a novel synthetic process for the preparation of 2-alkyl-1-((2′-substituted-biphenyl-4-yl)methyl)-imidazole, dihydroimidazole or benzimidazole derivatives such as 2-butyl-3-[[2′-[1-(triphenylmethyl)-1H-tetrazol-5-yl][1,1′-biphenyl]-4-yl]methyl]-1,3-diazaspiro[4.4]non-1-en-4-one, a key intermediate in the synthesis of irbesartan, or 4′-[(1,4′-dimethyl-2′-propyl-[2,6′-bi-1H-benzimidazol]-1′-yl)methyl]-[1,1′-biphenyl]-2-carboxylic acid, telmisartan.

### BACKGROUND OF THE INVENTION

Irbesartan and telmisartan together with other angiotensin II antagonists share a common structure consisting of 4,1′-disubstituted biphenyl where one substituent is preferably tetrazole or carboxy group and the other an alkyl substituent which is further substituted, preferably by at least one nitrogen atom containing heterocycle. The preferred angiotensin II antagonists in accordance with our invention are irbesartan, telmisartan, losartan, candesartan, olmesartan and valsartan and their derivatives. The most preferred are irbesartan and telmisartan.

2-Butyl-3-[[2′-[1-(triphenylmethyl)-1H-tetrazol-5-yl][1,1′-biphenyl]-4-yl]methyl]-1,3-diazaspiro[4.4]non-1-en-4-one (I) is a key intermediate for the synthesis of irbesartan, (2-butyl-3-[[2′-(1H-tetrazol-5-yl)[1,1′-biphenyl]-4-yl]methyl]-1,3-diazaspiro[4.4]non-1-en-4-one). Irbesartan is synthesized from compound with formula I, by removal of trityl group.

The synthesis of irbesartan is described, among other compounds, in U.S. Pat. Nos. 5,270,317 and 5,559,233. The synthesis therein disclosed proceeds by coupling the 2-butyl-1,3-diazaspiro[4.4]non-1-en-4-one with 4′-(bromomethyl)biphenyl-2-carbonitrile, followed by addition of azide to carbonitrile group or reacting the 1-aminocyclo-pentanecarboxamide with 4′-(aminomethyl)biphenyl-2-carbonitrile and valeric acid ortho-ester, followed by addition of azide.

WO 2004/072964 describes preparation of trityl irbesartan from ethyl 1-aminocyclo-pentanecarboxylate, (2′-(1-trityl-1H-tetrazol-5-yl)biphenyl-4-yl)methanamine and ethyl valerimidate methanesulfonic acid salt.

WO 2004/007482 describes process of making trityl irbesartan from 2-butyl-1,3-diazaspiro[4.4]non-1-en-4-one hydrochloride with 5-(4′-(bromomethyl)biphenyl-2-yl)-1-trityl-1H-tetrazole, in the two phase system, using phase transfer catalyst.

WO 2004/065383 disclosed the process of making trityl irbesartan from 2-butyl-3-(4′-bromophenyl)-1,3-diazaspiro[4.4]non-1-ene-4-one with 2-(1-trityl-1H-tetrazol-5-yl)phenylboronic acid, using conditions of Suzuki reaction, in the two phase system, in the presence of phase transfer catalyst.

EP 502314 disclosed the process of making telmisartan, among others, from (2′-carboxy-biphenyl-4-yl)methyl derivatives with nucleophilic leaving group and corresponding benzimidazole.

Suzuki coupling reaction has been used for building biphenylic part of the angiotensin II antagonist to which subsequently a heterocyclic part is constructed. This reaction usually needs an efficient catalyst, mainly a palladium catalyst. For industrial application there is a need for not only a safe, robust scalable process, but also for a cheap procedure. Palladium could play an expensive role in the synthetic scheme as a whole, so it is more suitable if enters in a later part of the synthetic pathway because fewer steps to the end of synthesis may mean less opportunity of losses due to incomplete reactions or isolations. Synthetic schemes like U.S. Pat. No. 5,270,317, U.S. Pat. No. 5,559,233, WO 2004/007482 and EP 502314, plan to initially build biphenylic part and subsequently condense it to the heterocyclic part. Thus the catalyst is used in the earlier step. Process in WO 2004/065383 builds biphenylic part into the imidazole structure per partes using a catalyst in the later step, but there is still a need for making boronic acid part in a less complicate species then on the phenyltetrazole part.

### DISCLOSURE OF THE INVENTION

The invention in one aspect represents a process for reaction of a compound with formula:

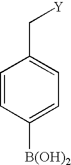

where Y represents leaving group, suitable for substitution with amino group, and is selected from the groups consisting of halogens, sulfonate esters; phosphate of phosphite esters, and chlorosulfites,
with optionally substituted imidazole derivative of formula

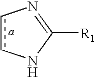

where a can be either single or double bond and said imidazole is selected from any one of those of formulas,

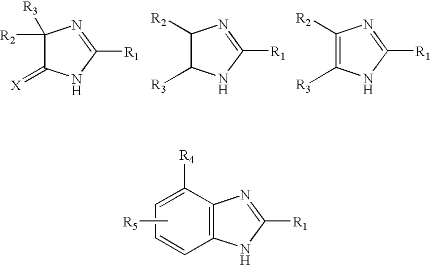

in which:
R₁ is a hydrogen, a C₁-C₆ alkyl which is unsubstituted or substituted by one or more halogen atoms, a C₂-C₆ alkenyl, a C₃-C₇ cycloalkyl, a phenyl, a phenylalkyl in which the alkyl is C₁-C₃, or a phenylalkenyl in which the alkenyl is C₂-C₃, said phenyl groups being substituted or polysubstituted by a halogen atom, a C₁-C₄ alkyl, a C₁-C₄ halogenoalkyl, C₁-C₄ polyhalogenoalkyl, a hydroxyl or a C₁-C₄ alkoxy group,
R₂ and R₃ are same or different and are each independently hydrogen or a group selected from halogen atoms, a C₁-C₆ alkyl which is unsubstituted or substituted by one or more hydroxyl groups or halogen atoms, a C₃-C₇ cycloalkyl, a C₁-C₄ alkoxy, a hydroxyl, an amino, an amino alkyl, a carboxyl, carboxyaldehyde, an alkoxycarbonyl in which the alkoxy is C₁-C₄, a cyano, a tetrazolyl,
or R₂ or R₃ form a group of formula —(CH₂)ₙ—O—CO—R₆ where n is an integer between 2 and 6; and R₆ is selected from group consisted of a C₁-C₆ alkyl which is unsubstituted or substituted by one or more halogen, nitro, cyano, hydroxyl or alkoxy group; phenyl which may be unsubstituted or substituted with (nitrooxy)methyl, alkoxymethyl, or halogen atom,
or R₂ and R₃ together form a group of the formula —(CH₂)ₙ—, where n is an integer between 2 and 11,
X is an oxygen or sulphur atom,
R₄ is hydrogen, or a group selected from halogen atoms, a C₁-C₆ alkyl which is unsubstituted or substituted by one or more halogen atoms, a C₃-C₇ cycloalkyl, a C₁-C₄ alkoxy,
R₅ represents a carboxylic group, benzimidazol-2-yl or 4,5,6,7-tetrahydro-benzimidazol-2-yl group, optionally substituted in the 1-position by C₁-C₆ alkyl or a cycloalkyl group, whilst the phenyl nucleus of one of the above-mentioned benzimidazole groups may additionally be substituted by a fluorine atom or by methyl or trifluoromethyl group,
   or their corresponding hydrohalides
   where a compounds presented with any of the formulae

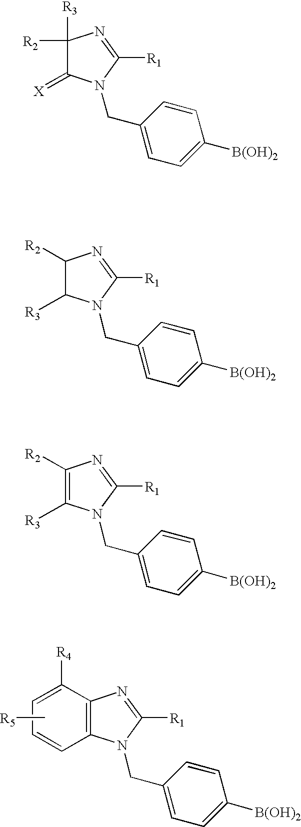

are prepared.

In another aspect the invention is embodied in new compounds: 4-[(2-butyl-4-oxo-1,3-diazaspiro[4.4]non-1-en-3-yl)methyl]phenylboronic acid, which can be used as an intermediate in the process for preparing irbesartan; 1-(4-boronobenzyl)-4-(2-hydroxypropan-2-yl)-2-propyl-1H-imidazole-5-carboxylic acid which can be used as an intermediate in the process for preparing olmesartan; 4-((2-butyl-4-chloro-5-(hydroxymethyl)-1H-imidazol-1-yl)methyl)phenylboronic acid and 4-((2-butyl-4-chloro-5-formyl-1H-imidazol-1-yl)methyl)phenylboronic acid, which can be used as intermediates in the process for preparing losartan; 3-(4-boronobenzyl)-2-ethoxy-3H-benzo[d]imidazole-4-carboxylic acid which can be used as an intermediate in the process for preparing candesartan and (S)-2-(N-(4-boronobenzyl)pentanamido)-3-methylbutanoic acid which can be used as an intermediate in the process for preparing valsartan. The aspects of the invention are also derivatives, preferably salts and protected forms of above compounds, which are preferably esters thereof.

In yet another aspect the invention provides for a use of 4-[(2-butyl-4-oxo-1,3-diazaspiro[4.4]non-1-en-3-yl)methyl]phenylboronic acid or 1-(4-boronobenzyl)-4-(2-hydroxypropan-2-yl)-2-propyl-1H-imidazole-5-carboxylic acid or 4-((2-butyl-4-chloro-5-(hydroxymethyl)-1H-imidazol-1-yl)methyl)phenylboronic acid or 4-((2-butyl-4-chloro-5-formyl-1H-imidazol-1-yl)methyl)phenylboronic acid or 3-(4-boronobenzyl)-2-ethoxy-3H-benzo[d]imidazole-4-carboxylic acid or (S)-2-(N-(4-boronobenzyl)pentanamido)-3-methylbutanoic acid in the preparation of angiotenzin II antagonists. The aspect of the invention is also the use of the salts and protected forms of above compounds.

In additional aspect the invention is a process where the compound prepared as described above or the compound obtainable as described above, but prepared by another process, is reacted with the compound of formula

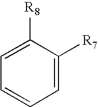

where R₈ is a halogen and R₇ is selected from the group consisting of an optionally protected tetrazole, cyano and carboxy group, to prepare an optionally substituted imidazole derivative of formula

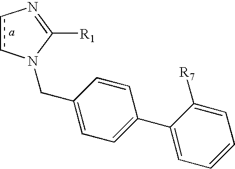

where a can be either single or double bond and said substituted imidazole is selected from any one of those of formulas:

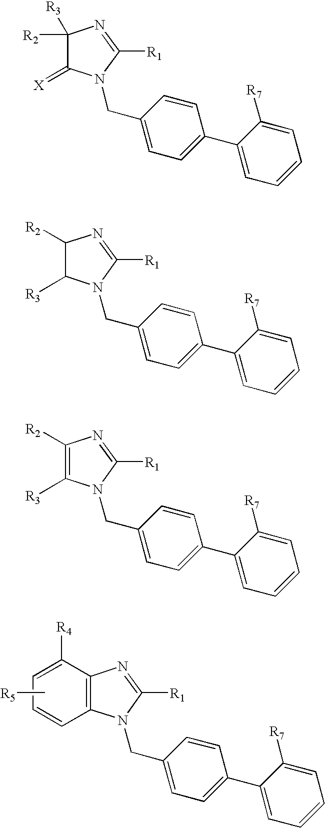

where R₁, R₂, R₃, R₄, R₅, and X and R₇ are as defined above. In an aspect of the invention any of the protecting groups are (if desired) subsequently removed and/or the obtained compound is subsequently converted into angotension II antagonist selected form the group consisting of irbesartan, telmisartan, losartan, candesartan, olmesartan, its salts and esters.

The aspect of the invention are also a pharmaceutical composition comprising a compound prepared as described above, preferably an angiotenzin II antagonist prepared from the above mentioned intermediate and/or its use in preparation of a medicament and/or use for manufacturing of a medicament for treating hypertension.

A specific embodiment of the invention is a process for the preparation of 2-butyl-3-[[2′-[1-(triphenylmethyl)-1H-tetrazol-5-yl][1,1′-biphenyl]-4-yl]methyl]-1,3-diazaspiro[4.4]non-1-en-4-one, which proceeds via novel intermediate, 4-[(2-butyl-4-oxo-1,3-diazaspiro[4.4]non-1-en-3-yl)methyl]phenylboronic acid which is prepared from 2-butyl-1,3-diazaspiro[4.4]non-1-en-4-one and 4-bromomethylphenyl boronic acid in DMF, in the presence of lithium bis(trimethylsilyl)amide at room temperature.

### DETAILED DESCRIPTION OF INVENTION

Our invention provides for synthesis of complicated heterocyclic system. In the synthetic scheme the catalyst enter into the synthesis in the late part when biphenyl moiety is constructed. Starting materials which enter into the biphenyl construction could be made from simple materials like 4-bromotoluene and 1-phenyltetrazole. Surprisingly the substance of the invention can be prepared from simple compounds by a short two step synthesis.

In the first step methylphenylboronic acid derivative is coupled with an imidazole derivative, preferably 2-butyl-1,3 diazaspiro[4.4]non-1-en-4-one or its analogue.

In the second step thus formed methylphenylboronic acid derivative is reacted with benzene derivative using conditions of Suzuki coupling, in the presence of palladium or nickel catalyst.

In a specific example of the process of our invention is depicted on Scheme 1.

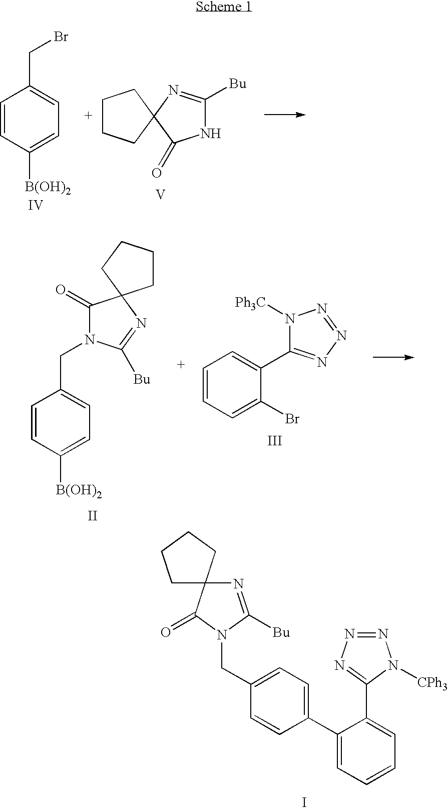

In accordance with our invention the methylphenylboronic acid derivative of the first step is presented with the formula below,

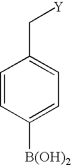

where Y represents leaving group, suitable for substitution with amino group, and is selected from the groups consisting of halogens, sulfonate esters such as tosyl, mesyl, brosyl, triflyl; phosphate of phosphite esters, or chlorosulfites, preferably Y is F, Cl, Br, I, tosyl, mesyl, brosyl or triflyl, more preferably bromo.

The preferable starting compound is thus 4-bromomethylphenyl boronic acid, which can be prepared according to the literature procedure (Snyder, H. R.; Reedy, A. J.; Lennarz, W. J. Amer. Chem. Soc. 1958, 80, 835).

The imidazole derivative 2-butyl-1,3-diazaspiro[4.4]non-1-en-4-one, or its analog if other angiotensin II antagonists besides irbesartan is being prepared, may enter reaction as a hydrochloride salt, or free base, which is prepared by neutralization using suitable base, preferably potassium of sodium hydroxide.

The imidazole derivative or its analog may be substituted imidazole, dihydroimidazole or benzimidazole and can be a compound selected from the group consisting of: 2-butyl-1,3-diazaspiro[4.4]non-1-en-4-one, 1,4′-dimethyl-2′-propyl-2,6′-bi(1H-benzo[d]imidazole); 2-butyl-5-chloro-1H-imidazole-4-carbaldehyde; (2-butyl-5-chloro-1H-imidazol-4-yl)methanol; 4-(2-hydroxypropan-2-yl)-2-propyl-1H-imidazole-5-carboxylic acid or it's protected derivatives. The imidazole derivative or its analog may be in from of salt or acid addition salt thereof, preferably hydrohalide most preferably hydrochloride salt.

The suitable imidazole analogs can be presented with general formula

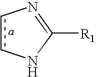

where a can be either single or double bond and can be further substituted, in preferred embodiment said imidazole is selected from any one of those of formulas, formulas below:

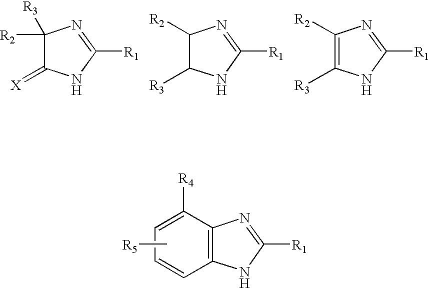

in which:
R₁ is a hydrogen, a C₁-C₆ alkyl which is unsubstituted or substituted by one or more halogen atoms, a C₂-C₆ alkenyl, a C₃-C₇ cycloalkyl, a phenyl, a phenylalkyl in which the alkyl is C₁-C₃, or a phenylalkenyl in which the alkenyl is C₂-C₃, said phenyl groups being substituted or polysubstituted by a halogen atom, a C₁-C₄ alkyl, a C₁-C₄ halogenoalkyl, C₁-C₄ polyhalogenoalkyl, a hydroxyl or a C₁-C₄ alkoxy group,
R₂ and R₃ are same or different and are each independently hydrogen or a group selected from halogen atoms, a C₁-C₆ alkyl which is unsubstituted or substituted by one or more hydroxyl groups or halogen atoms, a C₃-C₇ cycloalkyl, a C₁-C₄ alkoxy, a hydroxyl, an amino, an amino alkyl, a carboxyl, carboxyaldehyde, an alkoxycarbonyl in which the alkoxy is C₁-C₄, a cyano, a tetrazolyl,
or R₂ or R₃ form a group of formula —(CH₂)ₙ—O—CO—R₆ where n is an integer between 2 and 6; and R₆ is selected from group consisted of a C₁-C₆ alkyl which is unsubstituted or substituted by one or more halogen, nitro, cyano, hydroxyl or alkoxy group; phenyl which may be unsubstituted or substituted with (nitrooxy)methyl, alkoxymethyl, or halogen atom,
or R₂ and R₃ together form a group of the formula —(CH₂)ₙ—, where n is an integer between 2 and 11,
X is an oxygen or sulphur atom,
R₄ is hydrogen, or a group selected from halogen atoms, a C₁-C₆ alkyl which is unsubstituted or substituted by one or more halogen atoms, a C₃-C₇ cycloalkyl, a C₁-C₄ alkoxy,
R₅ represents a carboxylic group, benzimidazol-2-yl or 4,5,6,7-tetrahydro-benzimidazol-2-yl group, optionally substituted in the 1-position by C₁-C₆ alkyl or a cycloalkyl group, whilst the phenyl nucleus of one of the abovementioned benzimidazole groups may additionally be substituted by a fluorine atom or by methyl or trifluoromethyl group.

The following compounds selected from the above group of imidazole derivatives are especially preferred: 2-butyl-1,3-diazaspiro[4.4]non-1-en-4-one; 1,4′-dimethyl-2′-propyl-2,6′-bi(1H-benzo[d]imidazole); 2-butyl-5-chloro-1H-imidazole-4-carbaldehyde; (2-butyl-5-chloro-1H-imidazol-4-yl)methanol; 4-(2-hydroxypropan-2-yl)-2-propyl-1H-imidazole-5-carboxylic acid or it's protected derivatives.

Reaction is performed in the solvent selected from aprotic solvents such as N,N-dimethylformamide, N,N-dimethylacetamide, acetonitrile, tetrahydrofuran, dioxan, 1-methylpyrrolidinone, 1,3-dimethyl-3,4,5,6-tetrahydro-2(1H)-pyrimidone, dichloromethane, dimethylsulfoxide, toluene, benzene, or alcohols containing from one to six carbon atoms, or mixtures thereof; preferably N,N-dimethylformamide or N,N-dimethylacetamide, more preferably in N,N-dimethylformamide.

Reaction is performed in the presence of the base, selected from a group of organic bases such as lithium bis(trimethylsilyl)amide, sodium bis(trimethylsilyl)amide, hexamethyldisilazane, secondary amine, tertiary amine, sodium methoxide, potassium methoxide, sodium ethoxide, potassium ethoxide, or from a group of inorganic bases such as sodium hydride, sodium hydroxide, potassium hydroxide; preferable lithium bis(trimethylsilyl)amide or sodium hydride.

Temperature at which reaction is performed should be in range from −80 to 160° C., preferably from −10 to 25° C., more preferably at room temperature.

Preferable embodiment comprises a coupling of 2-butyl-1,3-diazaspiro[4.4]non-1-en-4-one and 4-bromomethylphenylboronic acid in N,N-dimethylformamide, in the presence of lithium bis(trimethylsilyl)amide at 25° C. for up to 48 hours, preferably up to 24 hours. Another preferable embodiment comprises a coupling of 2-butyl-1,3-diazaspiro[4.4]non-1-en-4-one hydrochloride and 4-bromomethylphenylboronic acid in N,N-dimethylformamide, in the presence of sodium hydride at room temperature for up to 24 hours, preferably up to 4 hours.

Another preferred embodiment comprises a coupling of 2′-propyl-1,4′-dimethyl-2,6′-bi(1H-benzo[d]imidazole) (V) and 4-bromomethylphenylboronic acid in N,N-dimethylformamide, in the presence of lithium bis(trimethylsilyl)amide at 25° C. (Scheme 2).

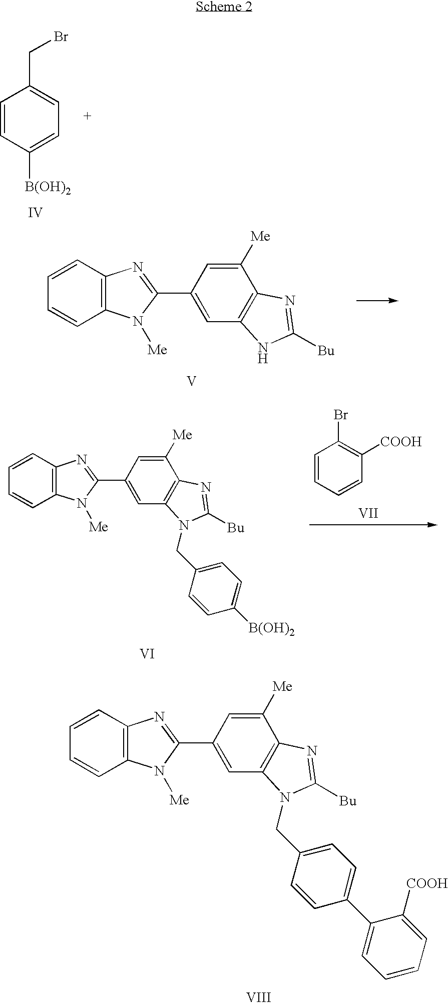

In the second step the compound obtained in first step is coupled with ortho substituted benzene derivative of formula

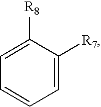

where one substituent (R₇) is a halogen, preferably bromo and the other (R₈) is an optionally protected tetrazole, cyano or carboxy group, or even a group which can be converted to tetrazole or carboxy, using conditions of Suzuki coupling, in the presence of palladium or nickel catalyst.

In a specific embodiment 4-[(2-butyl-4-oxo-1,3-diazaspiro[4.4]non-1-en-3-yl)methyl]phenylboronic acid (II) or its analog and 5-(2-bromophenyl)-1-(triphenylmethyl)-1H-tetrazole (III) are coupled using conditions of Suzuki coupling, in the presence of palladium or nickel catalyst.

The catalyst may be selected from the group consisting of palladium complexes and nickel complexes.

Preferable catalyst comprises a combination of palladium complex and triaryl phosphine. More preferable, catalyst is tetrakis(triphenylphosphine)palladium(0), neat or prepared in situ from palladium (II) acetate and triphenyl phosphine. Most preferably the catalyst is tetrakis(triphenylphosphine)palladium(0).

Reaction is performed in organic solvent selected from aprotic solvents such as N,N-dimethylformamide, N,N-dimethylacetamide, acetonitrile, tetrahydrofuran, dioxan, 1-methylpyrrolidinone, 1,3-dimethyl-3,4,5,6-tetrahydro-2(1H)-pyrimidone, dichloromethane, dimethylsulfoxide, toluene, benzene, or alcohols containing from one to six carbon atoms, or mixtures thereof, preferably in mixture of toluene, ethanol and water.

Reaction is performed in the presence of aqueous solution of sodium or potassium carbonate. Reaction is performed in the temperature interval from −80° C. to 160° C., preferably from 25 to 120° C., more preferably at the reflux temperature of the mixture. Preferably the reaction is performed in the mixture of toluene, ethanol and water

An embodiment represents the coupling of 4-[(2-butyl-4-oxo-1,3-diazaspiro[4.4]non-1-en-3-yl)methyl]phenylboronic acid (II) and 5-(2-bromophenyl)-1-(triphenylmethyl)-1H-tetrazole (III), in the presence of tetrakis(triphenylphosphine)palladium(0) and sodium carbonate. Preferably the reaction proceeds in mixture of degassed toluene and aqueous solution of sodium carbonate for several hours, preferably up to 24 hours, more preferably up to 6 hours at reflux temperature.

Another embodiment represents the coupling of 4′-[(1,4′-dimethyl-2′-propyl-[2,6′-bi-1H-benzimidazol]-1′-yl)methyl]phenylboronic acid (VI) and 2-bromobenzoic acid (VII) in the mixture of toluene, methanol and water, in the presence of tetrakis(triphenylphosphine)palladium(0) and sodium carbonate, at reflux temperature of the reaction mixture.

The following examples are offered to illustrate aspects of the present invention, and are not intended to limit or define the present invention in any manner.

### EXAMPLE 1

### Preparation of 4-[(2-Butyl-4-oxo-1,3-diazaspiro[4.4]non-1-en-3-yl)methyl]phenylboronic Acid (Compound of Formula II)

To a solution of 3.37 g of 2-butyl-1,3-diazaspiro[4.4]non-1-en-4-on hydrochloride in the 50 ml mixture of acetone/water 1:1, a solution of 0.58 g NaOH in 10 ml water was added. Then 40 ml water was added and extracted twice with 50 ml CH₂Cl₂. Combined organic phases were dried over anhydrous magnesium sulphate and concentrated, then dissolved in 30 ml DMF, cooled to 0° C. and stirred under inert atmosphere. 15 ml of 1M solution LiN(SiMe₃)₂ in THF was slowly added, and stirred at 0° C. for additional 20 min, then solution of 1.05 g compound of formula IV in 40 ml DMF was slowly added to the reaction mixture, which was then left to warm up to r.t. and stirred at r.t for 20-24 h. After that, 200 ml of ethyl acetate was added in washed twice with brine. Organic phase was dried over anhydrous magnesium sulphate, and then concentrated. After addition of diethyl ether, white precipitate was collected by filtration to give compound of formula II (1.93 g).

MS, (M+H)⁺=329 m/z. M.p.=107-134° C.

¹H NMR (300 MHz, DMSO-d₆): δ 0.76 (t, J=5.2, 3H), 1.22 (m, 2H), 1.44 (m, 2H), 1.65 (m, 2H), 1.82 (m, 6H), 2.28 (t, J=7.6, 2H), 4.66 (s, 2H), 7.08 (d, 8.0, 2H), 7.74 (d, J=8.0, 2H), 8.05 (s, 2H).

¹³C NMR (300 MHz, DMSO-d₆): δ 13.6, 21.5, 25.4 (2), 26.6, 27.5, 36.8 (2), 42.6, 75.8, 125.4 (2), 134.5 (2), 138.9, 161.1, 185.7.

### EXAMPLE 2

### Preparation of 4-[(2-Butyl-4-oxo-1,3-diazaspiro[4.4]non-1-en-3-yl)methyl]phenylboronic Acid (Compound of Formula II)

Suspension of 1.065 g of 2-butyl-1,3-diazaspiro[4.4]non-1-en-4-on hydrochloride in 25 ml DMF was cooled to 0° C., then 0.462 g of sodium hydride (60% suspension in mineral oil) was added and left to warm to r.t, then stirred at this temperature for two hours. Reaction mixture was then cooled to 0° C., and solution of 1.15 g of compound of formula IV in 10 ml DMF was slowly added. After allowing reaction mixture to warm up, it was stirred at r.t for 3-4 hours, and then 100 ml of ethyl acetate was added, and washed twice with brine. Organic phase was dried over magnesium sulphate, concentrated, and then after addition of diethyl ether, white precipitate was collected by filtration to give compound of formula II (0.89 g).

### EXAMPLE 3

### Preparation of 2-butyl-3-[[2′-[1-(triphenylmethyl)-1H-tetrazol-5-yl][1,1′-biphenyl]-4-yl]methyl]-1,3-diazaspiro[4.4]non-1-en-4-one (Compound of Formula I)

The mixture of 463 mg of 5-(2-bromofenil)-1-(triphenylmethyl)-1H-tetrazole (formula II) and 55 mg tetrakis(triphenylphosphine)palladium(0) was dissolved in 25 ml of degassed toluene, and stirred for 15 min under inert atmosphere. A solution of 235 mg sodium carbonate in 3 ml of water was added, followed by solution of 322 mg of compound of formula II in 8 ml of ethanol. The mixture was then refluxed under inert atmosphere for 5-6 h then cooled to r.t. Afterwards, 100 ml of dichloromethane was added, then organic phase passed through pad of silica, dried over anhydrous magnesium sulphate and concentrated. After addition of diethyl ether, trityl irbesartane (compound of formula I) crystallizes from reaction mixture (0.47 g).

MS, (M+H)⁺=671 m/z

¹H NMR (300 MHz, DMSO-d₆): δ 0.70 (t, J=7.3, 3H), 1.12 (m, 2H), 1.41 (m, 2H), 1.67 (m, 2H), 1.83 (m, 6H), 2.19 (t, J=7.5, 2H), 4.62 (s, 2H), 6.86 (t, J=7.3, 6H), 7.04 (m, 4H), 7.32 (m, 10H), 7.53 (m, 2H), 7.78 (d, J=7.7, 1H).

¹³C NMR (300 MHz, DMSO-d₆): δ 13.6, 21.4, 25.5 (2), 26.4, 27.4, 36.8 (2), 42.2, 75.8, 82.3, 125.7, 126.1 (2), 127.8 (6), 128.3 (3), 129.3 (2), 129.6 (6), 130.3, 130.4, 130.6, 135.8, 139.4, 140.8 (3), 141.1

### EXAMPLE 4

### Preparation of 4′-[(1,4′-dimethyl-2′-propyl-[2,6′-bi-1H-benzimidazol]-1′-yl)methyl]phenylboronic Acid (Compound of Formula VI)

A solution of 1.32 g 2′-propyl-1,4′-dimethyl-2,6′-bi(1H-benzo[d]imidazole) (V) in DMF (40 ml), under inert atmosphere (N₂) is cooled (0° C.), then 4.33 ml of 1M solution LiN(SiMe₃)₂ in THF was slowly added, stirred for another 30 min, then slowly solution of 0.93 g compound of formula IV in 15 ml DMF was added to the reaction mixture, which was then left to warm up to r.t. and stirred at r.t for 20-24 h. After that, 200 ml of ethyl acetate was added in washed twice with brine. Organic phase was dried over anhydrous magnesium sulphate, and then concentrated. After addition of diethyl ether, light brown precipitate was collected by filtration to give compound of formula VI (0.94 g).

MS, (M+H)⁺=439 m/z

¹H NMR (300 MHz, DMSO-d₆): δ 0.95 (t, J=7.5, 3H), 1.76 (m, 2H), 2.62 (s, 3H), 2.86 (t, J=7.8, 2H), 3.81 (s, 3H), 5.57 (s, 2H), 7.08 (d, J=8.1, 2H), 7.23 (m, 2H), 7.46 (s, 1H), 7.60 (m, 2H), 7.70 (s, 1H), 7.72 (d, J=8.1, 2H), 8.02 (s, 2H).

1. A process for preparing compounds selected from the group consisting of: 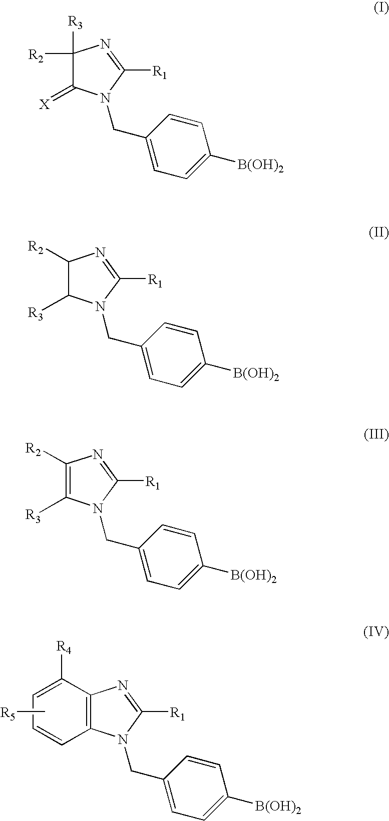
comprising reacting a compound of formula: 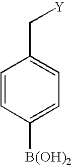
where Y represents a leaving group which is selected from the group consisting of: halogens, sulfonate esters, phosphate or phosphite esters, and chlorosulfites,
with optionally substituted imidazole of formula: 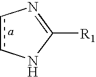
where a can be either a single or double bond, and said imidazole is selected from the group consisting of: 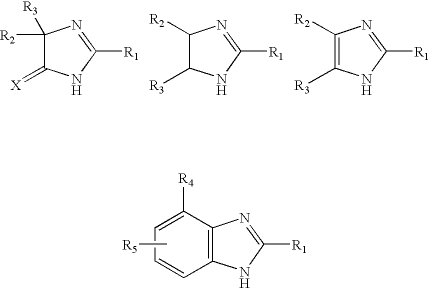
in which:
R₁ is a hydrogen, a C₁-C₆ alkyl optionally substituted by one or more halogen atoms, a C₂-C₆ alkenyl, a C₃-C₇ cycloalkyl, a phenyl, a phenylalkyl in which the alkyl is C₁-C₃, or a phenylalkenyl in which the alkenyl is C₂-C₃, where said phenyl group is optionally substituted by one or more substituents selected from the group consisting of: a halogen atom, a C₁-C₄ alkyl, a C₁-C₄ halogenoalkyl, C₁-C₄ polyhalogenoalkyl, a hydroxyl and a C₁-C₄ alkoxy group,
R₂ and R₃ are the same or different and each independently from each other is hydrogen or selected from the group consisting of: a halogen, a C₁-C₆ alkyl which is unsubstituted or substituted by one or more hydroxyl groups or halogen atoms, a C₃-C₇ cycloalkyl, a C₁-C₄ alkoxy, a hydroxyl, an amino, an amino alkyl, a carboxyl, a carboxyaldehyde, an alkoxycarbonyl in which the alkoxy is C₁-C₄, a cyano, and a tetrazolyl,
or R₂ or R₃ form a group of formula —(CH₂)ₙ—O—CO—R₆ where n is an integer between 2 and 6; and R₆ is selected from group consisting of: C₁-C₆ alkyl which is unsubstituted or substituted by one or more halogen, nitro, cyano, hydroxyl or alkoxy group; and phenyl which is unsubstituted or substituted with (nitrooxy)methyl, alkoxymethyl, or halogen atom,
or R₂ and R₃ together form a group of the formula —(CH₂)ₙ—, where n is an integer between 2 and 11,
X is an oxygen or sulfur atom,
R₄ is a hydrogen, or is selected from the group consisting of: halogen atoms, a C₁-C₆ alkyl which is unsubstituted or substituted by one or more halogen atoms, a C₃-C₇ cycloalkyl, and a C₁-C₄ alkoxy,
R₅ represents a carboxylic group, benzimidazol-2-yl or 4,5,6,7-tetrahydro-benzimidazol-2-yl group, optionally substituted in the 1-position by C₁-C₆ alkyl or a cycloalkyl group, and wherein the phenyl nucleus of one of the abovementioned benzimidazole groups may additionally be substituted by a fluorine atom or by a methyl or trifluoromethyl group,
or their corresponding hydrohalides.

2. A process according to claim 1 where Y is selected from the group consisting of: Br, Cl, F, I, tosyl, mesyl, brosyl and triflyl.

3. A process according to claim 1, where said imidazole is selected from the group consisting of: 2-butyl-1,3-diazaspiro[4.4]non-1-en-4-on, 1,4′-dimethyl-2′-propyl-2,6′-bi(1H-benzo[d]imidazole); 2-butyl-5-chloro-1H-imidazole-4-carbaldehyde; (2-butyl-5-chloro-1H-imidazol-4-yl)methanol; 4-(2-hydroxypropan-2-yl)-2-propyl-1H-imidazole-5-carboxylic acid, and salts thereof.

4. A process according to claim 1, where one of the reactants is 4-bromomethylphenylboronic acid.

5. A process according to claim 1, where said process is performed in an organic solvent selected from aprotic solvents selected from the group consisting of: N,N-dimethylformamide, N,N-dimethylacetamide, acetonitrile, tetrahydrofuran, dioxan, 1-methylpyrrolidinone, 1,3-dimethyl-3,4,5,6-tetrahydro-2(1H)-pyrimidone, dichloromethane, dimethylsulfoxide, toluene, benzene, alcohols containing from one to six carbon atoms, and mixtures thereof.

6. A process of claim 5, where said process is performed in N,N-dimethylformamide.

7. A process according to claim 1, where said process is performed in the presence of a base.

8. A process according to claim 7, where said process is performed in a presence of an organic base.

9. A process according to claim 8, where the organic base is selected from the group consisting of: lithium bis(trimethylsilyl)amide, sodium bis(trimethylsilyl)amide, hexamethyldisilazane, secondary amine, tertiary amine, sodium methoxide, potassium methoxide, sodium ethoxide, and potassium ethoxide.

10. A process according to claim 9, where said process is performed with lithium bis(trimethyl-silyl)amide.

11. A process according to claim 1, where said process is performed in a presence of an inorganic base.

12. A process according to claim 11, where the inorganic base is selected from the group consisting of sodium hydride, sodium hydroxide and potassium hydroxide.

13. A process according to claim 12, where the inorganic base is sodium hydride.

14. A process according to claim 1, where said process is performed in the temperature range from −80° C. to 160° C.

15. A process according to claim 14, where said process is performed in the temperature range from −10° C. to 25° C.

16. A compound selected from the group consisting of: 4-[(2-butyl-4-oxo-1,3-diazaspiro[4.4]non-1-en-3-yl)methyl]phenylboronic acid, 4′-[(1,4′-dimethyl-2′-propyl-[2,6′-bi-1H-benzimidazol]-1′-yl)methyl]phenylboronic acid, 1-(4-boronobenzyl)-4-(2-hydroxypropan-2-yl)-2-propyl-1H-imidazole-5-carboxylic acid, 4-((2-butyl-4-chloro-5-(hydroxymethyl)-1H-imidazol-1-yl)methyl)phenylboronic acid, 4-((2-butyl-4-chloro-5-formyl-1H-imidazol-1-yl)methyl)phenylboronic acid, 3-(4-boronobenzyl)-2-ethoxy-3H-benzo[d]imidazole-4-carboxylic acid, and (S)-2-(N-(4-boronobenzyl)pentanamido)-3-methylbutanoic acid.

17. A method of preparing angiotensin II antagonists, comprising using a compound selected from the group consisting of 4-[(2-butyl-4-oxo-1,3-diazaspiro[4.4]non-1-en-3-yl)methyl]phenylboronic acid, 4′-[(1,4′-dimethyl-2′-propyl-[2,6′-bi-1H-benzimidazol]-1′-yl)methyl]phenylboronic acid, 1-(4-boronobenzyl)-4-(2-hydroxypropan-2-yl)-2-propyl-1H-imidazole-5-carboxylic acid, 4-((2-butyl-4-chloro-5-(hydroxymethyl)-1H-imidazol-1-yl)methyl)phenylboronic acid, 4-((2-butyl-4-chloro-5-formyl-1H-imidazol-1-yl)methyl)phenylboronic acid, 3-(4-boronobenzyl)-2-ethoxy-3H-benzo[d]imidazole-4-carboxylic acid, and (S)-2-(N-(4-boronobenzyl)pentanamido)-3-methylbutanoic acid.

18. A process where the compound obtained according to claim 1 is reacted with the compound of formula 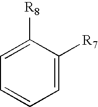
where R₈ is a halogen and R₇ is selected from the group consisting of an optionally protected tetrazole, cyano and carboxy group, to prepare an optionally substituted imidazole derivative of formula 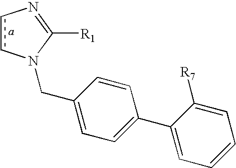
where a can be either single or double bond and said imidazole is selected from any one of the following: 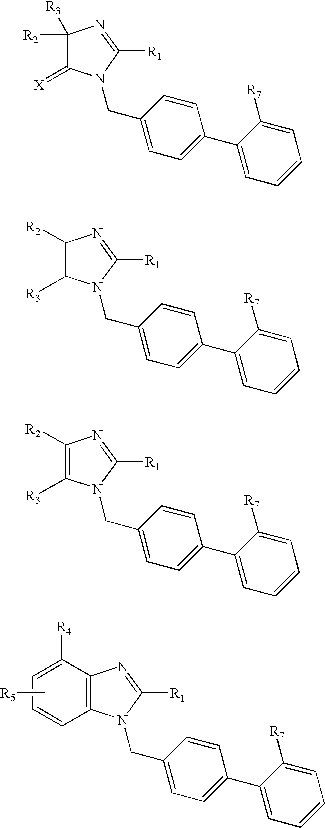
and R₁, R₂, R₃, R₄, R₅, and X are as defined in claim 1 and R₇ as defined above.

19. A process where the compound prepared according to claim 1 is subsequently reacted with a compound of formula 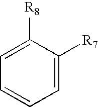
where R₈ is a halogen and R₇ is selected from the group consisting of an optionally protected tetrazole, optionally protected carboxy group and cyano group, to prepare an optionally substituted imidazole derivative of formula 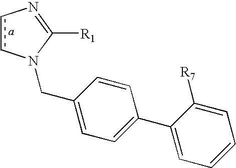
where a can be either single or double bond and said imidazole is selected from any one of the following: 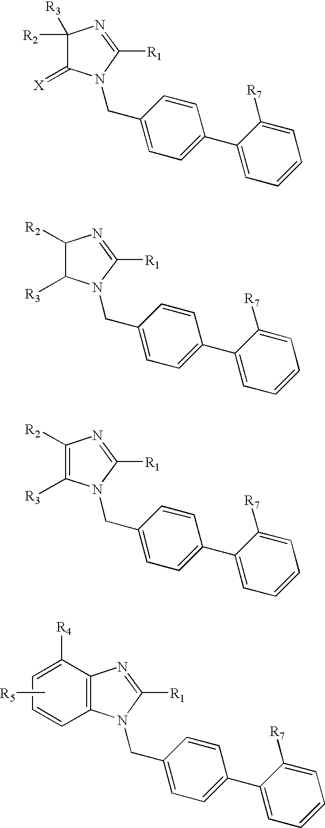
and R₁, R₂, R₃, R₄, R₅, and X are as defined in claim 1 and R₇ as defined above.

20. A process for preparing 2-butyl-3-[[2′-[1-(triphenylmethyl)-1H-tetrazol-5-yl][1,1′-biphenyl]-4-yl]methyl]-1,3-diazaspiro[4.4]non-1-en-4-one, comprising the step of reacting 4-[(2-butyl-4-oxo-1,3-diazaspiro[4.4]non-1-en-3-yl)methyl]phenylboronic acid with 5-(2-bromofenil)-1-(triphenylmethyl)-1H-tetrazole.

21. A process according to claim 18, wherein a catalyst selected from the group consisting of palladium complexes and nickel complexes is used.

22. A process according to claim 21, wherein the catalyst comprises a combination of a palladium complex and a triaryl phosphine.

23. A process according to claim 22, wherein the catalyst comprises a combination of a Pd(OAc)₂ and triphenyl phosphine.

24. A process according to claim 23, wherein the catalyst is tetrakis(triphenylphosphine)palladium(0), prepared in situ from Pd(OAc)₂ and triphenyl phosphine.

25. A process according to the claim 21, where catalyst is tetrakis(triphenyl-phosphine)palladium(0).

26. A process according to claim 18, where said process is performed in solvent where the solvent comprises one or more solvents selected from the group consisting of: N,N-dimethylformamide, N,N-dimethylacetamide, acetonitrile, tetrahydrofuran, dioxan, 1-methylpyrrolidinone, 1,3-dimethyl-3,4,5,6-tetrahydro-2(1H)-pyrimidone, dichloromethane, dimethylsulfoxide, toluene, benzene, alcohols containing from one to six carbon atoms, and water.

27. A process of claim 26, where said solvent comprises one or more of the solvents selected from the group consisting of toluene, ethanol, and water.

28. A process according to claim 18 characterized in that it is performed in the presence of an inorganic base selected from potassium carbonate or sodium carbonate.

29. A process according to claim 18, where said process is performed in the temperature range from −80° C. to 160° C.

30. A process according to claim 29, where said process is performed in the temperature range from 25° C. to 120° C.

31. A process according to claim 30, where said process is performed at the reflux temperature of the mixture of the solvents.

32. A process according to claim 18 comprising a subsequent step in which any of the protecting groups are removed.

33. A process according to claim 18 where the obtained compound is subsequently converted into an angotension II antagonist selected from the group consisting of: irbesartan, telmisartan, losartan, candesartan, olmesartan and salts and esters thereof.

34. A pharmaceutical composition comprising a compound prepared according to claim 18.

35. (canceled)

36. The pharmaceutical composition according to claim 34, wherein said composition comprises one or more of the compounds of formula (I), (II), (III), and (IV), in an amount effective for treating hypertension.
